(19) United States
(12) Patent Application Publication
(10) Pub. No.: US 2005/0209335 A1
(43) Pub. Date: Sep. 22, 2005

(54) Methods for use of TARGRETIN in organ transplant rejection, autoimmune diseases and cancer

(76) Inventor: Donald Tsai, Merion Station, PA (US)

Correspondence Address: Licata & Tyrrell P.C., 66 East Main Street, Marlton, NJ, 08053, US

(21) Appl. No.: 11/084,954

(22) Filed: Mar. 21, 2005

Related U.S. Application Data
(60) Provisional application No. 60/554,753, filed on Mar. 19, 2004.

(51) Int. Cl. A61K 31/192

(52) U.S. Cl. 514/569

(57) ABSTRACT

Methods are provided for treatment of graft versus host disease and other autoimmune diseases, organ transplant rejection, acute myeloid leukemia and T-cell non-Hodgkin's lymphoma other than cutaneous T-cell lymphoma in patients by administration of TARGRETIN. Also provided are methods for increasing neutrophil count and boosting the immune system of patients by administration of TARGRETIN and methods for enhancing efficacy of a cancer chemotherapeutic agent by co-administration of the cancer chemotherapeutic agent with TARGRETIN.

This patent application claims the benefit of priority from U.S. Provisional Application Ser. No. 60/554,753, filed Mar. 19, 2004, which is herein incorporated by reference in it entirety.

### FIELD OF THE INVENTION

The present invention relates to methods for use of TARGRETIN in the treatment of patients suffering from autoimmune diseases or disorders, organ transplant rejection, acute myeloid leukemia and T-cell lymphomas other than cutaneous T-cell lymphoma. The present invention also relates to methods for use of TARGRETIN in increasing neutrophil count in a patient, thereby boosting the immune system of patients in need thereof. In addition, methods for use of TARGRETIN in combination with other cancer chemotherapeutic agents to increase efficacy of the cancer treatment are provided.

### BACKGROUND OF THE INVENTION

TARGRETIN [4-((3,5,5,8,8-pentamethyl-5,6,7,8-tetrahydro-2-mapthyl)ethenyl)benzoic acid)], also known as bexarotene and LGD1069, is a retinoid X receptor (RXR) ligand. This compound, developed by Ligand Pharmaceuticals, Inc., was the first retinoid X receptor selective, antitumor retinoid to enter clinical trials (Hurst R. E. Current Opinion in Investigational Drugs 2000 1 (4):514-23).

TARGRETIN has been approved by the Food and Drug Administration for the treatment of cutaneous T-cell lymphoma (Cohen et al. Oncologist 2001 6 (1):4-11).

Topical cream formulations as well as oral formulations of TARGRETIN are available.

Results from Phase III clinical trials of topical TARGRETIN therapy for patients with refractory or persistent early stage cutaneous T-cell lymphoma are reported by Heald et al. in Journal of Academy of Dermatology 2003 49 (5):801-15. Optimization of TARGRETIN therapy for cutaneous T-cell lymphoma is discussed by Talphur et al. in Journal of American Academy of Dermatology 2002 47 (5):672-84.

The effects of TARGRETIN in psoriasis have also been examined (Franssen et al. British Journal of Dermatology 2003 149 (3):506-12; Smit et al. Skin Pharmacology and Applied Skin Physiology 2003 16 (4):237-41).

TARGRETIN has also been suggested to be an effective palliative treatment for lymphomatoid papulosis, a clonal T cell proliferation with large cell histology, a chronic course and an increased risk of lymphoma (Krathen et al. Dermatology 2003 206 (2):142-7).

TARGRETIN has been reported to be efficacious as a chemopreventative and chemotherapeutic agent in rat N-nitroso-N-methylurea (NMU)-induced mammary carcinomas (Bischoff et al. Cancer Res. 1998 58:479-484). US20030013766 discloses use of TARGRETIN in treating a host with anti-estrogen resistant breast cancer. TARGRETIN has also been reported to decrease squamous cell carcinoma of the head and neck proliferation in part by interfering with TGF-alpha/EGFR autocrine signaling (Song et al. Cancer Research 2001 61 (15):5919-25). In addition a multi-institutional phase I/II trial of oral TARGRETIN in combination with cisplatin and vinorelbin in previously untreated patients with advance non-small-cell lung carcinoma yielded acceptable phase II response rates with better-than expected survival (Khuri et al. Journal of Clinical Oncology 2001 19 (10):2626-37). TARGRETIN has also be suggested to an effective therapeutic agent for uterine leiomyoma that may inhibit tumor growth and consequently alleviate the symptoms associated with this disease (Gamage et al. Journal of Pharmacology and Experimental Therapeutics 2000 295 (2):677-81).

In addition TARGRETIN has been disclosed for use in treatment of mycosis fungoide, a low grade myoproliferative disorder of skin-honing CD4+ lymphocytes that may produce patches, plaques, tumors, erythroderma and ultimately systemic dissemination (Smith, B. D. and Wilson, L. D. Oncology 2003 17 (10):1419-28).

Administration of TARGRETIN as a single daily oral dose of 450 mg/m² in capsule form was reported to be ineffective against melanoma (Bedikian et al. Oncology Reports 2000 7 (4):883-6).

It is well known that retinoic acids can cause all forms of acute myeloid leukemia cells to differentiate from leukemia cells into normal immune cells in vitro. However, doses required for in vitro results are toxic in humans.

Analogues of TARGRETIN where the carboxylic acid function was replaced by an N,N-dimethyl-S-aryl carbamate or N-N-dimethyl-O-arylthiocarbamate were shown to be more potent than TARGRETIN in inducing apoptosis in leukemia HL-60 cells (Winum et al. Bioorganic and Medicinal Chemistry Letters 2002 12 (24):3529-32).

### SUMMARY OF THE INVENTION

An object of the present invention is to provide a method for treating an autoimmune disease or disorder or organ transplant rejection in a patient which comprises administering to the patient an effective amount of TARGRETIN.

Another object of the present invention is to provide a method for treating patients with acute myeloid leukemia or T-cell lymphomas other that cutaneous T-cell lymphoma which comprises administering to the patient an effective amount of TARGRETIN.

Another object of the present invention is to provide a method for enhancing efficacy of a cancer chemotherapeutic agent in a patient which comprises administering to the patient the cancer chemotherapeutic agent in combination with TARGRETIN.

Yet another object of the present invention is to provide a method for increasing neutrophil count in a patient thereby boosting the immune system in patients in need thereof which comprises administering to the patient an effective amount of TARGRETIN.

### DETAILED DESCRIPTION OF THE INVENTION

It has now been found that TARGRETIN is useful in treating autoimmune diseases or disorders, organ transplant rejection and various cancers other than cutaneous T-cell lymphoma. Further, it has also now been found that TARGRETIN is useful in increasing neutrophils, thereby boosting the immune system in patients in need thereof. In addition, is has now been found that TARGRETIN enhances efficacy of cancer chemotherapy upon co-administration with the cancer chemotherapy. The present invention thus provides new methods for treating patients suffering from an automimmune disease or disorder, rejection following organ transplant or cancer, enhancing efficacy of a co-administered cancer chemotherapeutic agent and increasing neutrophil count thereby boosting the immune system of a patient in need thereof which comprise administering an effective amount of TARGRETIN.

By the terms “treat”, “treating” and “treatment” as used herein it is meant to include curing of the disease or condition or any improvement in the disease state or condition of a patient, as well as an alleviation or lessening in severity of a symptom or symptoms of the disease state or condition in a patient.

Further, by “effective amount” as used herein, it is meant a concentration of TARGRETIN administered by a selected route and dosing schedule which lessens or decreases severity of a symptom of the disease state or condition from which the patient is suffering and/or a concentration of TARGRETIN which increases neutrophil count in a patient and/or a concentration of TARGRETIN which increases the efficacy of a co-administered cancer chemotherapeutic agent. Such amounts can be routinely determined by those skilled in the art based upon the teachings herein as well as what is known in the art about TARGRETIN with respect to treatment of cutaneous T-cell lymphoma. Amounts of TAGRETIN exemplified herein to be effective at lessening or decreasing severity of a symptom of the disease, increasing neutrophil count, and/or enhancing efficacy of a co-administered cancer chemotherapeutic agent, without unwanted side effects include oral administration of 2-3 capsules a day or 150 to 225 mg a day. Additional dosing regimes well-tolerated and demonstrated to be effective in ongoing clinical trials include administration of TARGRETIN at 100 to 150 mg per meter square of body surface area. Further, the Food and Drug Administration's approved dose for TARGRETIN in the treatment of cutaneous T-cell lymphoma is 300 mg/M². It is expected that similar dosages will also be effective in treating autoimmune diseases or disorders, and in particular graft versus host disease, as well as organ transplant rejection. As this drug has been demonstrated to be well tolerated, it is expected that higher doses than those exemplified herein can be administered as well.

Both oral and topical formulations of TARGRETIN are available and approved for use by the Food and Drug Administration in cutaneous T-cell lymphoma. As demonstrated herein, these topical and oral formulations are also useful in methodologies of the present invention. Topical and oral administration are preferred routes of administration for TARGRETIN in the methodologies of the present invention. However, as will be understood by one of skill in the art upon reading this disclosure, other routes resulting in an effective amount of TARGRETIN being administered to a patient can be used.

Graft versus host disease, and many other severe autoimmune disease as well as organ transplant rejection are routinely treated with tacrolimus and steroid class agents. Topical administration of tacrolimus or picolimus as well as topical and/or oral administration of steroids is also typically used in treating chronic eczema dermatitis, a chronic skin inflammatory condition related to T-cell activity. These are known as T-cell stunning agents. However, tacrolimus has many long term side effects which can be very detrimental, if not fatal. Detrimental side effects of long-term steroid use are also well established.

TARGRETIN attacks and down-regulates T-cells. This is believed to be its mechanism of action in the treatment of cutaneous T-cell lymphoma. TARGRETIN has been found to be an effective substitute for tacrolimus and steroids in the treatment of eczema dermatitis. The inventor now believes that TARGRETIN is useful in other conditions and/or diseases where tacrolimus or steroids are used. Specifically, the inventor now believes that TARGRETIN is useful in graft versus host disease as well as other severe autoimmune disease and in organ transplant rejection.

Thus, in one embodiment, the present invention provides methods for treating graft versus host disease or another severe autoimmune disease or organ transplant rejection in a patient by administering to the patient an effective amount of TARGRETIN. Preferably the TARGRETIN is administered orally or topically.

Acute myeloid leukemia is a very aggressive disorder with poor prognosis. In general, only one in five patients suffering from this disease are actually cured. Very often, despite treatment with very aggressive chemotherapy, patients with AML relapse and at the time of relapse suffer from immune system collapse. Further treatment results in further injury to the immune system and rarely controls the leukemia. Thus, patients die either from infection or from progressive leukemia.

Administration of TARGRETIN, however, has now been found to be effective in treating patients with acute myeloid leukemia (AML). In these studies, five patients with relapsed refractory AML were treated with TARGRETIN monotherapy.

The first patient had failed induction chemotherapy, had fungal pneumonia, and active leukemia with no immune system. This patient was sent home under hospice care, but given TARGRETIN to take orally in a pill formulation. Within a few months, this patient no longer needed hospice care and had returned to work. It is believed that upon administration of TARGRETIN, this patient was able to maintain an immune system, thereby clearing the fungal pneumonia and preventing further fatal infections. The patient's leukemia was kept under control for nine months, at which point the patient developed progressive leukemia.

The second patient was a heart transplant patient who developed AML and failed induction chemotherapy as well as a second line of chemotherapy. With active leukemia and no immune system he was placed on TARGRETIN. Within one week of the TARGRETIN treatment, the patient's neutrophil count and/or immune system was rising and his leukemia count was dropping. Upon escalation of the TARGRETIN dose it was observed that his neutrophil count continued to increase and the leukemia count dropped. This increase in neutrophil count and decrease in leukemia count continued with each dose escalation until the patient was taking six TARGRETIN capsules a day. At that point the patient developed a pulmonary syndrome very similar to that seen in patients taking trans retinoic acid (ATRA) for AML type M3. This syndrome is a cell differentiation disorder wherein leukemia cells differentiate into normal immune cells. While this syndrome is good in that it boosts the immune system while decreasing the leukemia count, cytokines released by the differentiating neutrophils can cause a pulmonary syndrome. At six TARGRETIN capsules a day the second patient developed a syndrome exactly like that observed in ATRA administration. TARGRETIN treatment was stopped and within one week the neutrophil count dropped from 1500 to 200 and the pulmonary syndrome resolved. TARGRETIN treatment was then restarted at three capsules daily and the neutrophil count returned to 600. This patient continued treatment for an additional 9 months at which time he developed an infection and died. At the time of death his leukemia was still well under control.

The third patient had failed induction therapy and was suffering from active AML with disseminated intravascular coagulation (DIC), a blood clotting disorder relating to active leukemia. This patient was completely bed-ridden. It was believed that she could not tolerate further chemotherapy and was placed on TARGRETIN. Unexpectedly, her DIC resolved within four days of starting TARGRETIN and her leukemia count dropped. She also developed a small number of immune cells and was able to return to her home where her physical condition continued to improve. This patient lived for approximately 4.5 months before she developed progressive leukemia. At that point she declined further therapy and expired.

The fourth patient had raging AML. This patient was placed on TARGRETIN but expired within 3 days of starting treatment. It is believed that the TARGRETIN treatment may have been started too late in the course of this patient's disease to provide an effect.

The fifth patient treated with TARGRETIN was suffering from a relapse after already having undergone an allogeneic bone marrow transplant, one of the most aggressive treatments possible for AML. Upon initiation of TARGRETIN treatment, this patient mounted an immune system response. This patient ultimately expired, however, from complications relating to his bone marrow transplant.

Thus, 4 out of 5 patients suffering from terminal AML, when treated with TARGRETIN exhibited a positive response evidenced by an increase in neutrophil count and/or a decrease in leukemia count.

TARGRETIN has also been found to be effective in treating patients suffering from subsets of non-Hodgkin's T-cell lymphomas other than cutaneous T-cell lymphoma.

Specifically, TARGRETIN was administered to a patient suffering from a post-transplant lymphoproliferative disorder type lymphoma. This is a T-cell non-Hodgkin's lymphoma related to organ transplantation. This patient had horrific lymphoma with involvement of his cerebral spinal fluid. Previous treatment with chemotherapy had failed. With TARGRETIN, however, this patient has been in remission for four months.

TARGRETIN was administered to a second patient suffering from a rare type of T-cell lymphoma, an angiocentric T-cell lymphoma. Previous attempts to treat the disease in this patient by bone marrow transplant had failed. With TARGRETIN, however, this patient has been in remission for three months.

Thus, as demonstrated herein, administration of TARGRETIN is effective in treating patients suffering from acute myeloid leukemia as well as T-cell non-Hodgkin's lymphomas other than cutaneous T-cell lymphoma. Accordingly, the present invention also provides methods for treating patients suffering from these cancers which comprise administering an effective amount of TARGRETIN to the patient. Preferably the TARGRETIN is administered topically or orally.

As also demonstrated herein, administration of TARGRETIN increased the neutrophil count in patients, thereby boosting their immune system. Thus, the present invention also provides methods for increasing neutrophil count in a patient which comprise administering to the patient an effective amount of TARGRETIN. This increase in neutrophil count provides a boost to a patient's immune system. Thus, such methods are particularly useful in patients suffering from a weakened immune system. Preferably in these methods the TARGRETIN is administered orally or topically.

TARGRETIN has also now been found to act synergistically with conventional cancer chemotherapeutics thereby enhancing efficacy of the cancer chemotherapeutic treatment. In these experiments, two patients with refractory acute lymphocytic leukemia were treated with TARGRETIN in combination with low dose chemotherapy. In both patients efficacy of the treatment was significantly greater than expected and out of proportion to the intensity of the chemotherapy. Without be limited to any specific theory, the enhanced efficacy of treatment of the chemotherapeutic agent upon co-administration with TARGRETIN may be related to the action of TARGRETIN on lymphocytes, which may effect pathways that make a cancer cell more susceptible to chemotherapy.

Thus, the present invention also provide methods for enhancing efficacy of a cancer chemotherapeutic agent which comprise administering to a patient suffering from cancer a cancer chemotherapeutic agent in combination with TARGRETIN. Preferably the patient is administered low dose chemotherapy in combination with TARGRETIN. By “combination” as used herein it is meant to be inclusive of, but not limited to, dosing regimes wherein the cancer chemotherapeutic agent and TARGRETIN are administered simultaneously via the same route and simultaneously via different routes, as well as dosing regimes wherein TARGRETIN is administered before the cancer chemotherapeutic agent or after the cancer chemotherapeutic agent via the same or different dosing route.

1. A method for treating an autoimmune disease or disorder or organ transplant rejection in a patient comprising administering to the patient an effective amount of TARGRETIN.

2. The method of claim 1 wherein the TARGRETIN is administered orally or topically.

3. The method of claim 1 wherein the autoimmune disease is graft versus host disease.

4. The method of claim 1 wherein the patient is treated for organ transplant rejection.

5. A method for treating acute myeloid leukemia and T-cell non-Hodgkin's lymphoma other than cutaneous T-cell lymphoma in a patient comprising administering to the patient an effective amount of TARGRETIN.

6. The method of claim 5 wherein the TARGRETIN is administered orally or topically.

7. The method of claim 5 wherein the patient is suffering from acute myeloid leukemia.

8. The method of claim 5 wherein the patient is suffering from T-cell non-Hodgkin's lymphoma other than cutaneous T-cell lymphoma.

9. A method for enhancing efficacy of a cancer chemotherapeutic agent in a patient comprising administering to the patient a cancer chemotherapeutic agent in combination with TARGRETIN.

10. The method of claim 9 wherein the TARGRETIN is administered orally or topically.

11. A method for increasing neutrophil count in a patient comprising administering to the patient an effective amount of TARGRETIN.

12. The method of claim 11 where the patient is suffering from a weakened immune system.

13. The method of claim 11 wherein the TARGRETIN is administered orally or topically.
